(19) United States
(12) Patent Application Publication
(10) Pub. No.: US 2010/0306866 A1
(43) Pub. Date: Dec. 2, 2010

(54) Adropin deficient mice and uses thereof

(76) Inventor: Andrew A. Butler, Jupiter, FL (US)

Correspondence Address: Alan F. Feeny, Esq., Biomeasure, Incorporated, 27 Maple Street, Milford, MA, 01757-3650, US

(21) Appl. No.: 12/460,020

(22) Filed: Jul. 10, 2009

Related U.S. Application Data
(60) Provisional application No. 61/134,620, filed on Jul. 11, 2008.

(51) Int. Cl. A01K 67/027

(52) U.S. Cl. 800/18

(57) ABSTRACT

Mice lacking expression of the Enho gene provide useful tools in the study of the Enho gene and to investigate possible treatments for glucose, lipid and energy metabolism.

This application claims benefit of U.S. Provisional patent application No. 61/134,620, filed Jul. 11, 2008.

### TECHNICAL FIELD

This invention pertains to methods and materials related to making and using transgenic rodents with a genomic disruption affecting the expression of ADROPIN, a secreted protein that is involved in the regulation of lipid metabolism and energy homeostasis in response to dietary nutrient composition.

### BACKGROUND

Energy homeostasis is a complex biological process by which an organism coordinates energy intake with substrate metabolism and nutrient composition of the diet. Obesity and insulin resistance are two common disorders of energy homeostasis which result from an organism's failure to balance and adapt energy homeostasis, particularly under conditions of abundant calorie-dense food and reduced physical activity-based energy expenditure (Hill, J. A., Endocrine Reviews, 2006, 27:750-761).

In many portions of the world, and in particular in industrialized nations where labor-intensive industries have been superseded, calorie-dense food is easily obtained in non-limiting quantities. As such, obesity is an increasingly prevalent global disease that, according to some experts, has reached epidemic proportions. Current estimates suggest that at least 50% of the global population is either overweight or obese. The significance of this for health is that obesity, and particularly abdominal obesity, is often accompanied by other conditions such as insulin resistance, dyslipidemia, hepatic steatosis and hypertension.

Insulin resistance (also referred to as hyperinsulinemia) is a condition in which insulin-stimulated glucose uptake is reduced in both skeletal muscle and fat (Reaven, G. M., Physiol. Rev., 1995, 75:473-486). Hyperinsulinemia is initially successful in suppressing liver glucose output; however the deleterious effects of the increased insulin offset the gains associated with maintaining normal blood glucose levels (Reaven, G. M., Physiol. Rev., 1995, 75:473-486). Hyperinsulinemia is thought to be a factor in a cluster of metabolic abnormalities, including hypertension, non-alcoholic fatty liver disease and coronary heart disease (Reaven, G. M., Physiol. Rev., 1995, 75:473-486).

A metabolic state conducive to the development of insulin resistance is thought to result from an imbalance of caloric intake with oxidative metabolism (Ravussin, E. et al., Ann. NY Acad. Sci., 2002, 967:363-378; Lowell, B. B., et al., Science, 2005, 307:384-387). Studies suggest that reduced mitochondrial function in muscle may be a factor in the development of insulin resistance associated with obesity (Lowell, B. B., et al., Science, 2005, 307:384-387;Sutton, G. M., et al., Endocrinology, 2006, 147:2183-2196.). Stimulation of energy expenditure and suppression of appetite both result in improved glucose metabolism in mouse models of obesity and type-2 diabetes. Defining a common mechanism explaining insulin resistance has been difficult because of the complexity of the insulin receptor signaling system and the fact that many factors contribute to the development of this disorder.

Insulin resistance typically precedes type-2 diabetes. In type-2 diabetes, the β-cells of the pancreas fail to produce sufficient insulin to compensate for insulin resistance; this results in a state of persistent hyperglycemia (Biddinger, S. B. et al., Ann. Rev. Physiol., 2006, 68:123-58). The mechanisms linking obesity to insulin resistance and type-2 diabetes are poorly understood, however hyperglycemia and hyperlipidemia are both side effects of, and causative agents in, the pathophysiology of type-2 diabetes. Glucotoxicity and lipotoxicity further promote insulin resistance and type-2 diabetes due to suppression of insulin action and secretion from the β-cells.

There is also an association between the disorders of insulin resistance and lipid metabolism in response to dietary nutrition. Studies of several mouse models of obesity and metabolic disorders suggest that the link between insulin resistance and dysregulated triglyceride metabolism is complex and involves both peripheral and central factors.

### SUMMARY OF THE INVENTION

ADROPIN is a secreted peptide that is encoded by a gene highly expressed in liver and central nervous system and which is involved in regulating energy homeostasis and lipid metabolism in response to dietary nutrient composition. ADROPIN [derived from the Latin root “aduro” (to set fire to) and “pinquis” (fats or oils)] is encoded by the “Energy Homeostasis Associated” transcript (gene symbol: Enho (previously referred to as Swir1); see WO 2007/019426 incorporated herein in its entirety; Kumar K G, et al., Adropin is a secreted in the liver, Enho mRNA is regulated by both energy status and dietary nutrient content and levels of Enho mRNA are altered in obese mice. Transgenic over-expression of the open reading frame encoding ADROPIN from the Enho gene or systemic ADROPIN treatment is sufficient to attenuate components of the metabolic distress associated with diet-induced obesity ((DIO); see WO 2007/019426 incorporated herein in its entirety; Kumar K G, et al., Adropin is a secreted peptide involved in energy homeostasis and lipid metabolism, 2008, submitted to Cell Metabolism, incorporated herein in its entirety).

This invention is based, in part, on the discovery that transgenic mice whose genomes contain a disruption in a nucleic acid encoding an ADROPIN polypeptide are resistant to insulin. Homozygous mutant mice exhibit glucose intolerance on a high fat diet as well as increased adiposity at an early stage of development as compared to wild-type counterparts. No significant difference of food intake is observed in homozygous mutants; the increased adiposity observed is thus attributed to a change in metabolism rather than diet. Moreover, glucose intolerance and insulin resistance are independent of weight gain and increased adiposity, suggesting that ADROPIN effects on obesity and glucose homeostasis are independent. Heterozygous mutants exhibit a similar phenotype of glucose intolerance and insulin resistance without exhibiting any body weight or adipose mass phenotype.

These results suggest that ADROPIN is a critical modulator of energy homeostasis, functioning to affect insulin resistance and lipid metabolism in response to dietary nutrition. As a result, such transgenic mice provide a model useful for studying the biological or biochemical roles of ADROPIN. Such transgenic mice also provide a model useful for studying the role of various drugs or other therapeutic treatments in the study of energy homeostasis, obesity, lipid metabolism, insulin resistance, diabetes, particularly type-2 diabetes, non-alcoholic fatty liver disease, Syndrome X and associated complications, hypertension, blood glucose levels and metabolism, trigylceride metabolism and the like.

The invention is not limited to transgenic mice; any non-human mammal may be used in the practice of the invention. Exemplary mammals include, but are not limited to, rodents such as rats, mice or guinea pigs, farm animals such as pigs, sheep, goats, horses, cattle, fowl and the like.

In one embodiment, the present invention provides a transgenic rodent in which native Enho gene function has been disrupted or “knocked out”. In one aspect of this embodiment, the transgenic rodent is fertile and can transfer this trait to progeny mice. In another aspect of this embodiment, the levels of expression of Enho have been reduced by at least about 50%, at least about 60%, at least about 70%, at least about 80%, at least about 90%, at least about 95%, at least about 98%, at least about 99% or even about 100% as compared to a wild-type rodent. In another aspect of this embodiment, the levels of expression of ADROPIN have been reduced by about at least 50%, about at least 60%, about at least 70%, about at least 80%, about at least 90%, about at least 95%, about at least 98%, about at least 99% or even about 100% as compared to a wild-type rodent. In a preferred aspect of this embodiment, the transgenic rodent is a rat and the levels of expression of Enho and ADROPIN are reduced by at least about 90% as compared to a wild-type rat. In another preferred aspect of this embodiment, the transgenic rodent is a mouse and the levels of expression of Enho and ADROPIN are reduced by at least about 90% as compared to a wild-type mouse.

In one embodiment, the invention provides a transgenic mouse, the genome of which comprises a homozygous disruption of the endogenous Enho gene and wherein the transgenic mouse lacks wild-type levels of ADROPIN peptide activity. The level of ADROPIN peptide may be so reduced as to be undetectable. This mouse is referred to as a “knockout mouse” or “KO mouse” or “Enho−/−.” In one aspect of this embodiment, the disruption is in an intron of the endogenous Enho gene. In a further aspect, the disruption is a deletion of an intron or a portion of an intron. In yet a further aspect, the disruption is a point mutation. In another aspect of this embodiment, the disruption is in an exon of the endogenous Enho gene. In a further aspect, the disruption is a deletion of an exon or a portion of an exon of the Enho gene. In yet a further aspect, the disruption is a point mutation. In a preferred aspect, all or a portion of exon 2 is deleted. In another embodiment, the disruption of the endogenous Enho gene is prepared using Cre-lox technology.

In yet another embodiment, the invention provides a transgenic mouse exhibiting insulin resistance as a result of a homozygous disruption of the endogenous Enho gene in the mouse genome and where the transgenic mouse lacks ADROPIN peptide activity.

In yet another embodiment, the invention provides a transgenic mouse exhibiting glucose intolerance as a result of a homozygous disruption of the endogenous Enho gene in the mouse genome and where the transgenic mouse lacks ADROPIN peptide activity. The glucose intolerant transgenic mice exhibit this phenotype when fed either a normal or a high fat diet.

In yet another embodiment, the invention provides a transgenic mouse exhibiting increased adiposity as a result of a homozygous disruption of the endogenous Enho gene in the mouse genome and where the transgenic mouse lacks ADROPIN peptide activity.

In yet another embodiment, the invention provides a transgenic mouse exhibiting non-alcoholic fatty acid liver disease as a result of a homozygous disruption of the endogenous Enho gene in the mouse genome and where the transgenic mouse lacks ADROPIN peptide activity.

In yet another embodiment, the invention provides a transgenic mouse exhibiting increased expression of the Pparg gene in adipose tissue as a result of a homozygous disruption of the endogenous Enho gene in the mouse genome and where the transgenic mouse lacks ADROPIN peptide activity.

In yet another embodiment, the invention provides a transgenic mouse exhibiting increased expression of the Pparg gene in adipose tissue as a result of a homozygous disruption of the endogenous Enho gene in the mouse genome and where the transgenic mouse lacks ADROPIN peptide activity.

In another embodiment, the invention provides a method of studying ADROPIN peptide function and activity which utilizes a transgenic mouse, the genome of which comprises a homozygous disruption of the endogenous Enho gene and wherein the transgenic mouse lacks ADROPIN peptide

In another embodiment, the invention provides a method of studying insulin resistance, the regulation of insulin and/or the regulation of glucose in a mammal which utilizes a transgenic mouse, the genome of which comprises a homozygous disruption of the endogenous Enho gene and wherein the transgenic mouse lacks ADROPIN peptide

In another embodiment, the invention provides a method of studying the regulation of lipids biochemistry which utilizes a transgenic mouse, the genome of which comprises a homozygous disruption of the endogenous Enho gene and wherein the transgenic mouse lacks ADROPIN peptide.

In another embodiment, the invention provides a method of producing antibodies, particularly monoclonal antibodies, which utilizes a transgenic mouse, the genome of which comprises a homozygous disruption of the endogenous Enho gene and wherein the transgenic mouse lacks ADROPIN peptide. The antibodies may be raised against native ADROPIN, fragments of native ADROPIN, analogs of ADROPIN or fragments of analogs of ADROPIN. The Enho −/− mouse is also used to produce hybridomas expressing monoclonal antibodies against native ADROPIN, fragments of native ADROPIN, analogs of ADROPIN or fragments of analogs of ADROPIN.

In one embodiment, the invention provides a transgenic mouse, the genome of which comprises a heterozygous disruption of the endogenous Enho gene and wherein the transgenic mouse lacks wild-type levels, i.e., reduced levels as compared to wild-type levels, of ADROPIN peptide activity. For purposes of this application, this mouse is referred to as a “het-knockout mouse” or “het-KO mouse” or “Enho +/−.” In one aspect of this embodiment, the disruption is in an intron of the endogenous Enho gene. In a further aspect, the disruption is a deletion of an intron or a portion of an intron. In yet a further aspect, the disruption is a point mutation. In another aspect of this embodiment, the disruption is an exon of the endogenous Enho gene. In a further aspect, the disruption is a deletion of an exon or a portion of an exon of the Enho gene. In yet a further aspect, the disruption is a point mutation. In a preferred aspect, all or a portion of exon 2 is deleted. In another embodiment, the disruption of the endogenous Enho gene is prepared using Cre-lox technology.

In yet another embodiment, the invention provides a transgenic mouse exhibiting insulin resistance as a result of a heterozygous disruption of the endogenous Enho gene in the mouse genome and where the transgenic mouse lacks wild-type levels of ADROPIN peptide activity.

In yet another embodiment, the invention provides a transgenic mouse exhibiting glucose intolerance as a result of a heterozygous disruption of the endogenous Enho gene in the mouse genome and where the transgenic mouse lacks wild-type levels of ADROPIN peptide activity. The glucose intolerant transgenic mice exhibit this phenotype when fed either a normal or a high fat diet.

In yet another embodiment, the invention provides a transgenic mouse exhibiting increased adiposity as a result of a heterozygous disruption of the endogenous Enho gene in the mouse genome and where the transgenic mouse lacks wild-type levels of ADROPIN peptide activity.

In yet another embodiment, the invention provides a transgenic mouse exhibiting non-alcoholic fatty acid liver disease as a result of a heterozygous disruption of the endogenous Enho gene in the mouse genome and where the transgenic mouse lacks wild-type levels of ADROPIN peptide activity.

In yet another embodiment, the invention provides a transgenic mouse exhibiting increased expression of the Pparg gene in adipose tissue as a result of a heterozygous disruption of the endogenous Enho gene in the mouse genome and where the transgenic mouse lacks wild-type levels of ADROPIN peptide activity.

In yet another embodiment, the invention provides a transgenic mouse exhibiting increased expression of the Pparg gene in adipose tissue as a result of a heterozygous disruption of the endogenous Enho gene in the mouse genome and where the transgenic mouse lacks wild-type levels of ADROPIN peptide activity.

In another embodiment, the invention provides a method of studying ADROPIN peptide function and activity which utilizes a transgenic mouse, the genome of which comprises a heterozygous disruption of the endogenous Enho gene and wherein the transgenic mouse lacks wild-type levels of ADROPIN peptide

In another embodiment, the invention provides a method of studying insulin resistance, the regulation of insulin and/or the regulation of glucose in a mammal which utilizes a transgenic mouse, the genome of which comprises a heterozygous disruption of the endogenous Enho gene and wherein the transgenic mouse lacks wild-type levels of ADROPIN peptide

In another embodiment, the invention provides a method of studying the regulation of lipids biochemistry which utilizes a transgenic mouse, the genome of which comprises a heterozygous disruption of the endogenous Enho gene and wherein the transgenic mouse lacks wild-type levels of ADROPIN peptide.

### DETAILED DESCRIPTION OF THE INVENTION

Recent studies have reported that staggering numbers of people world wide are overweight and suffering a wide variety of serious and expensive health problems. According the World Health Organization (as reported in Kouris-Blazos, A. et al., Asia Pac. J. Clin. Nutr., 2007, 16:329-338), an estimated 1 billion people throughout the world are overweight and an estimated 300 million of these are obese. An estimated 22 million children under the age of 5 are severely overweight and in the European Union alone, the number of children who are overweight is expected to rise by 1.3 million children per year (Kosti, R. I. et al., 2006, Cent. Eur. J. Public Health, 14:151-159).

Obesity, as defined by the Statistical Bulletin provided by the Metropolitan Life Insurance Co., (1959, 40:1), is a condition in which a person is approximately 20-25% over normal body weight. Alternatively, an individual is considered obese if the person has a body mass index of greater than 25% over normal or greater than 30% over normal with risk factors (see Bray, G. A., et al., Diabetes/Metabolism Review, 1988, 4:653-679 or Flynn, et al., Proc. Nutritional Society, 1991, 50:413). One of the main causes for obesity is the consumption of a high caloric diet (Riccardi, et al., Clin. Nutr., 2004, 23:447-456).

Diabetes is a chronic, debilitating disease afflicting many overweight and obese people. It is estimated that 20.8 million people in the United States alone have diabetes and that greater than 6 million more additional cases remain undiagnosed (Cornell, S. A., J. Manag. Care Pharm., 2007, 13:S11-5). Type-2 diabetes (also referred to herein as type II diabetes) is a chronic disease characterized by insulin resistance, impaired insulin secretion and hyperglycemia. Worldwide, type II diabetes is believed to affect approximately 171 million people, imparting numerous microvascular and macrovascular complications resulting in morbidity and mortality (Mudaliar, S., Indian J. Med. Res., 2007, 125:275-296).

Insulin resistance, also referred to as reduced insulin sensitivity, is a condition in which the amount of insulin needed to clear glucose from the blood of a subject is increased as compared to the amount of insulin needed to clear the same amount of glucose from the blood of a normal, non-insulin sensitive subject. Insulin resistance is regarded as the main link between obesity and type II diabetes (see Obici, et al., J. Clin. Inv., 2001, 108:1079-1085 and references therein). It is known that rats fed a high fat diet show an increase in body weight (diet-induced obesity or DIO) and a decrease in insulin sensitivity. Such DIO rats provide an animal model in which to study the mechanisms of insulin resistance due to obesity (see for example Banno, et al., FEBS letters, 2007, 581:1131-1136).

The size and weight of adipose tissues are increased in DIO rats and it is thought that the accompanying hypertrophy of adipocytes leads to changes in the release of adipocytokines such as leptin and adiponectin which are known to regulate insulin sensitivity; it is thought that morphological changes in adipose tissue as well as changes in plasma levels of adipocytokines are among the causes of insulin resistance in such obese rats (summarized in Banno, et al., FEBS letters, 2007, 581:1131-1136 and references therein).

Leptin acts in the hypothalamus and hindbrain to suppress appetite and, through stimulation of the autonomic nervous system, increases oxidative metabolism in skeletal muscle (Elmquist, J. K., et al., J. Comp. Neurol., 2005, 493:63-71; Asilmaz, E., et al., J. Clin. Invest., 2004, 113:414-424; Morton, G. J., et al., J. Clin. Invest., 2005, 115:703-710; Coppari, R., et al., Cell Metabolism, 2005, 1:63-72; Minokoshi, Y., et al., Nature, 2002, 415:339-343). However, leptin can also improve hepatic insulin sensitivity independently of marked effects on food intake or body weight (Asilmaz, E., et al., J. Clin. Invest., 2004, 113:414-424).

Many factors contribute to the development of insulin resistance, ranging from simple over-ingestion of high calorie food to the molecular complexities of the insulin receptor (IR) signaling system. Tyrosine phosphorylation of two adaptor proteins, IRS1 and IRS2, is a critical early step in the stimulation of glucose uptake by insulin (Araki, E., et al., Nature, 1994, 372:186-190; Withers, D. J., et al., Nature, 1998, 391:900-904; Kido, Y., et al., J. Clin. Invest., 2000, 105:199-205; Previs, S. F., et al., J. Biol. Chem., 2000, 275:38990-38994). IRS1 and IRS2 have no intrinsic enzymatic activity and are thought to function as part of a molecular scaffold that facilitates the formation of protein complexes with kinase, phosphatase or ubiquitin ligase functions (White, M. F., Am. J. Physiol. Endocrinol. Metab., 2002, 283:E413-422). Stimulation of phosphoinositide 3′ kinase (PI3K) by association with the insulin receptor signaling system is a critical step in insulin-stimulated glucose uptake. Activation of the p110 catalytic subunit of PI3K activates the lipid kinase domain, which phosphorylates phosphatidylinositol-4,5-bisphosphate. Activation of PI3K is necessary for full stimulation of glucose uptake by insulin, although other pathways might also be involved (White, M. F., Am. J. Physiol. Endocrinol. Metab., 2002, 283:E413-422).

As discussed previously, there is also a close association between the disorders of insulin resistance and lipid metabolism in response to dietary nutrition. The liver receives nutrients from the digestive system through the hepatic portal vein and regulates the postprandial processing and trafficking of carbohydrate and lipids. The central pathophysiological features of the dyslipidemia associated with insulin resistance and type-2 diabetes are increased secretion of plasma triglycerides in very low density lipoproteins from the liver along with reduced high density lipoprotein cholesterol.

Increased secretion of plasma triglycerides (TG) in very low density lipoproteins (VLDL) by the liver and reduced high density lipoprotein (HDL) cholesterol have been found to contribute to cardiovascular disease (Biddinger, S. B., et al., Cell Metab., 2008, 7:125-134; Petersen, K. F., et al., Proc. Natl. Acad. Sci. USA, 2007, 104:12587-12594; Petersen, K. F., et al., J. Clin. Invest., 2002, 109:1345-135; Oral et al., New Engl. J. Med., 2002, 346:570-578). Increased circulating TG are hydrolyzed into free fatty acids (FFA) which are taken up by peripheral tissues including the liver, leading to ectopic accumulation of fatty acids in the liver (hepatic steatosis; also known as non-alcoholic fatty liver disease (NAFLD)).

Clearly, the link between insulin resistance and dysregulated triglycerides is complex and involves both peripheral and central factors. For example, it has been shown that cross-talk between white adipose tissue (WAT) and other key insulin-target tissues plays a central role in lipid homeostasis and maintaining insulin sensitivity (Scherer, P. E., Diabetes, 2006, 55:1537-1545; Sharma, A. M. et al., J. Clin. Endocrinol. Metab., 2007, 92:386-395). The storage of energy in the TG pool of WAT protects against steatosis by preventing the deposition of fat in other tissues, particularly liver, skeletal muscle and pancreas (Morino, K., et al., Diabetes, 2006, 55Supp12:S9-S15). Steatosis is a factor in the development of, and is a physiological consequence of, insulin resistance in obesity (Morino, K., et al., Diabetes, 2006, 55Supp12:S9-515; Perlemuter, G., et al., Nat. Clin. Pract. Endocrinol. Metab. 2007, 3:458-469; Petersen, K. F., et al., Proc. Natl. Acad. Sci. USA, 2007, 104:12587-12594). Adipocytes also secrete factors (adipokines) that regulate lipid metabolism by acting as paracrine factors within WAT and as endocrine factors acting on the liver, muscle and central nervous system (CNS) (Scherer, P. E., Diabetes, 2006, 55:1537-1545). Adipocytes secrete adiponectin, which promotes insulin sensitivity by stimulating fat oxidation in liver and muscle (Scherer, P. E., Diabetes, 2006, 55:1537-1545; Sharma, A. M. et al., J. Clin. Endocrinol. Metab., 2007, 92:386-395) and by promoting the storage of TG preferentially in WAT (Kim, J. Y., et al., J. Clin. Invest., 2007, 117:2621-2637). With obesity, WAT secretes factors that contribute to insulin resistance, such as resistin and pro-inflammatory cytokines (Scherer, P. E., Diabetes, 2006, 55:1537-1545; Sharma, A. M. et al., J. Clin. Endocrinol. Metab., 2007, 92:386-395).

ADROPIN is a secreted peptide that is abundant in liver and the central nervous system that has recently be shown to be involved in regulating energy homeostasis and lipid metabolism in response to dietary nutrient composition. In C57BL/6J mice, a negative correlation was found in the hepatic expression of ADROPIN mRNA with fasting glucose levels. The expression of ADROPIN in the hypothalamus also declined with obesity and insulin resistance (see WO 2007/019426 incorporated herein in its entirety; Kumar K G, et al., Adropin is a secreted peptide involved in energy homeostasis and lipid metabolism, 2008, submitted to Cell Metabolism, incorporated herein in its entirety).

ADROPIN is also known to down regulate the expression of Peroxisome proliferator-activated receptor gamma (Pparg) in mice (Kumar K G, et al., Adropin is a secreted peptide involved in energy homeostasis and lipid metabolism, 2008, submitted to Cell Metabolism, incorporated herein in its entirety). Pparg is a transcription factor which regulates genes involved in fatty acid metabolism and the uptake and incorporation of fatty acids in the triglyceride storage depot (Sharma, A. M. et al., J. Clin. Endocrinol. Metab., 2007, 92:386-395).

Using an adenovirus expressing the native form of ADROPIN, it has been shown that increasing the expression of Enho mRNA encoding the ADROPIN protein in mouse models of obesity with high cholesterol and triglycerides is effective at reducing both cholesterol and triglycerides toward normal levels. The ability of excess ADROPIN to reduce triglyceride and total cholesterol were observed in three different mouse strains (Lep^{ob}/Lep^{ob}, KKA^{y}, and C57BL/6J). In some mice, an improvement in insulin sensitivity was observed in a trend of lower fasting insulin and glucose and an improvement in glucose tolerance test results. The latter observations suggested that ADROPIN may also be effective at improving insulin sensitivity in an obese, insulin-resistant patient (see WO 2007/019426 incorporated herein in its entirety; Kumar K. G., et al., Adropin is a secreted peptide involved in energy homeostasis and lipid metabolism, 2008, submitted to Cell Metabolism, incorporated herein in its entirety).

Additionally, the expression of a key gene involved in lipogenesis (fatty acid synthase) and FAS protein levels were reduced by ADROPIN adenoviral treatment in Lep^{ob}/Lep^{ob} mice. Mice infected with recombinant adenovirus expressing ADROPIN lost more weight during an overnight fast, suggesting an impaired ability to reduce metabolic rate to compensate during fasting (see WO 2007/019426 incorporated herein in its entirety; Kumar K. G., et al., Adropin is a secreted peptide involved in energy homeostasis and lipid metabolism, 2008, submitted to Cell Metabolism, incorporated herein in its entirety).

Transgenic strains of mice were created on C57BL/6J and FVB/NJ backgrounds which over express the Enho open reading frame, using the open reading frame encoding adropin from the Enho DNA sequence (SEQ ID NO:1) controlled by the human β-actin promoter which is expressed in all tissues (see WO 2007/019426 incorporated herein in its entirety; Kumar K. G., et al., Adropin is a secreted peptide involved in energy homeostasis and lipid metabolism, 2008, submitted to Cell Metabolism, incorporated herein in its entirety). Female FVB/NJ mice over expressing ADROPIN had a significant reduction in fat mass and a higher metabolic rate as determined by measuring oxygen consumption (VO2). FVB/NJ and C57BL/6J mice over expressing ADROPIN exhibit protection from diet-induce obesity and from metabolic disorders associated with obesity induced by high fat diet such as hyperinsulinemia, glucose intolerance and hepatic steatosis.

As with the mice infected with recombinant adenovirus expressing ADROPIN, FVB/NJ Enho transgenic mice exhibited exaggerated weight loss during a fast; weight loss under such conditions is believed to be associated with a higher metabolic rate. A component of ADROPIN anti-diabetic action may therefore involve stimulation of pathways involved in oxidative metabolism. That is, ADROPIN may improve the metabolic profile of obese, insulin resistant individuals partially through normalizing the balance of kJ consumption with kJ expended through effects on physical activity, basal metabolic rate, or a combination of both.

Melanocortin receptor knockout mice have also proven useful for investigating the link between obesity and insulin resistance. Melanocortins are a family of regulatory peptides which are formed by post-translational processing of pro-hormone pro-opiomelanocortin (POMC; 131 amino acids in length). POMC is processed into three classes of hormones; the melanocortins, adrenocorticotropin hormone, and various endorphins (e.g. lipotropin) (Cone, et al., Recent Prog. Horm. Res., 1996, 51:287-317; Cone et al., Ann. N.Y. Acad. Sci., 1993, 31:342-363).

Five melanocortin receptors (MC-R) have been characterized to date. These include melanocyte-specific receptor (MC1-R), corticoadrenal-specific ACTH receptor (MC2-R), melacortin-3 (MC3-R), melanocortin-4 (MC4-R) and melanocortin-5 receptor (MC5-R).

Two melanocortin receptors expressed in areas of the central nervous system are involved in energy homeostasis. Targeted deletion of the neuronal melanocortin-4 receptor (MC4R) gene in mice (Mc4r−/− or Mc4rKO mice) causes obesity and hyperinsulinemia, and is also associated with increased hepatic lipogenic gene expression and hepatic steatosis. Mice deficient for another neuronal melanocortin receptor (Mc3r−/− or Mc3rKO mice) develop a similar degree of obesity to Mc4r−/− mice when fed a high fat diet, but do not exhibit the same level of insulin resistance, hyperlipidemia and increased hepatic steatosis. Both Mc3rKO and Mc4rKO mice exhibit an exaggerated diet-induced obesity, however the deterioration of insulin sensitivity in Mc4rKO is more rapid and severe (31,32). In severely insulin resistant and glucose intolerant Mc4rKO and Leptin-deficient (Lep^{ob}/Lep^{ob}) mice, ADROPIN was found to be reduced 10-fold. In contrast, in obese Mc3rKO mice, which are moderately glucose intolerant but exhibit a normal response to insulin, there was a 30-40% reduction in the expression of ADROPIN protein.

What is unknown in the art, and addressed by the instant invention, are the characteristics and features of mice deficient in Enho expression and ADROPIN protein.

As used herein, an “effective amount” of a compound, protein or peptide of interest is any amount which delivers a measurable effect. The effect may be measured by biochemical, chemical or biological means, by monitoring genotypic and/or phenotypic characteristics or even by feedback provided by a subject receiving the compound, protein or peptide of interest. For example, an effective amount of ADROPIN protein or peptide is an amount that decreases the level of insulin resistance or of dyslipidemia, or that prevents, delays or reduces the incidence of the onset of type-2 diabetes in obese insulin resistant patients by a statistically significant degree.

As used herein, “statistical significance” is determined as the P<0.05 level, or by such other measure of statistical significance as is commonly used in the art for a particular type of experimental determination.

The term “ADROPIN” used herein and in the claims refers to the protein ADROPIN (SEQ ID NO:2), its functional peptides (e.g., ADROPIN³⁴⁻⁷⁶ (SEQ ID NO:3)), derivatives and analogs. The terms “derivatives” and “analogs” are understood to be proteins that are similar in structure to ADROPIN and that exhibit a qualitatively similar effect to the unmodified ADROPIN. The term “functional peptide” refers to a piece of the ADROPIN protein that still binds to the ADROPIN receptor or is able to activate changes inside body cells, e.g., adipocytes or hepatocytes.

The administration of ADROPIN, its functional peptides, its analogs and derivatives in accordance with the present invention may be used to reverse insulin resistance and dyslipidemia, to delay onset of type-2 diabetes in obese insulin resistant subjects, and to prevent or delay onset of obesity. These compounds can also be used as therapeutic or diagnostic agents for hypercholesterolemia, hypertriglyceridemia, insulin resistance, obesity, and diabetes.

As used herein “wild-type” refers to the usual state of existence for an organism with an unaltered genotype as compared to an organism harboring genetic alterations. In this invention, a “wild-type” C57BL/6J mouse is also referred to as Enho +/+indicating that the mouse contains two functional copies of the wild-type Enho gene.

The term “therapeutically effective amount” as used herein refers to an amount of ADROPIN protein, a fragment, a derivative or analog thereof sufficient to increase body energy expenditure, to decrease serum triglyceride, to decrease serum cholesterol, to decrease hyperlipidemia, and/or to decrease insulin resistance to a statistically significant degree (p<0.05). The dosage ranges for the administration of ADROPIN protein are those that produce the desired effect. Generally, the dosage will vary with the age, weight, condition, and gender of the patient. A person of ordinary skill in the art, given the teachings of the present specification, may readily determine suitable dosage ranges. The dosage can be adjusted by the individual physician in the event of any contraindications. In any event, the effectiveness of treatment can be determined by monitoring body metabolism, body weight, serum glucose, triglyceride levels, and/or cholesterol levels by methods well known to those in the field. Moreover, ADROPIN can be applied in pharmaceutically acceptable carriers known in the art.

### BRIEF DESCRIPTION OF THE DRAWINGS

FIG. 1 illustrates the constructs and targeting strategy used in the preparation of Enho knockout mice.

FIG. 2 illustrates the strategy employed to generate Enho knockout mice in which expression is affected in all cell types.

FIG. 3 illustrates the differences in percent fat mass (% FM) in young Enho −/− male and female mice fed standard rodent chow at an early age (<9 weeks).

FIG. 4 shows the effect of the loss of Enho mRNA upon the expression of select liver genes in female mice aged 8-9 weeks.

FIG. 5 shows the increased expression of Pparg gene expression in the adipose tissue in female mice aged 8-9 weeks.

FIG. 6 illustrates the effect of the loss of Enho mRNA upon fat mass, fat-free mass and overall bodyweight of male transgenic mice fed a high fat diet for 6 weeks.

FIG. 7 illustrates the effect of the loss of Enho mRNA upon fasting serum insulin, glucose and triglyceride of male transgenic mice fed standard rodent chow or high fat diet for 6 weeks.

FIG. 8 illustrates the effect of the loss of Enho mRNA upon glucose clearance in male mice after 4 weeks on high fat diet following administration of glucose (2 g/kg by intraperitoneal injection) or insulin (1U/kg by intraperitoneal injection).

FIG. 9 illustrates the effect of loss of Enho mRNA on the development of fatty liver in mice fed a high fat diet for 6 weeks.

FIG. 10 illustrates the effect of the loss of Enho mRNA on energy expenditure and spontaneous locomotory activity in male mice.

FIG. 11 illustrates the effect of the loss of Enho mRNA on substrate metabolism as measured by the Respiratory Exchange Ratio (RER) in male mice on a low fat or high fat diet.

FIG. 12 illustrates the effect of the loss of Enho mRNA on fatty acid oxidation in muscle lysates from male mice fed low fat diet.

### EXAMPLES

Provided herein are experimental methods useful in the practice of the present invention. The examples are in no way meant to be limiting to the practice of the invention. The skilled artisan would know that other methods may be employed to generate the transgenic mice of the invention and that other means of testing may be employed to determine the effect of the genetic disruption or to study the effect of compounds administered to the transgenic mice of the invention.

### Example 1

### Generation of B6.Cg-Enho^{loxP-(frt-Neo-frt)ORF-loxP} Mice

A conditional targeting vector designed to insert a segment of DNA containing (a) a selection cassette used to identify embryonic stem (ES) cell clones positive for insertion of foreign DNA flanked by Frt sites (Frt-Neo^{r}-Frt) and (b) LoxP sites flanking the Frt-Neo^{r}-Frt sequence and the adropin open reading frame was constructed by recombination-mediated genetic engineering within SW106 cells (Liu P., et al., Genome Research, 2003, 13:476-484). Isogenic DNA containing the Enho gene was retrieved from bacterial-artificial chromosome (BAC) clone RP23-10007 from a C57B1/6 BAC genomic library (BACPAC Resources Center at Children's Hospital Oakland Research Institute, Oakland, Calif.) via gap repair.

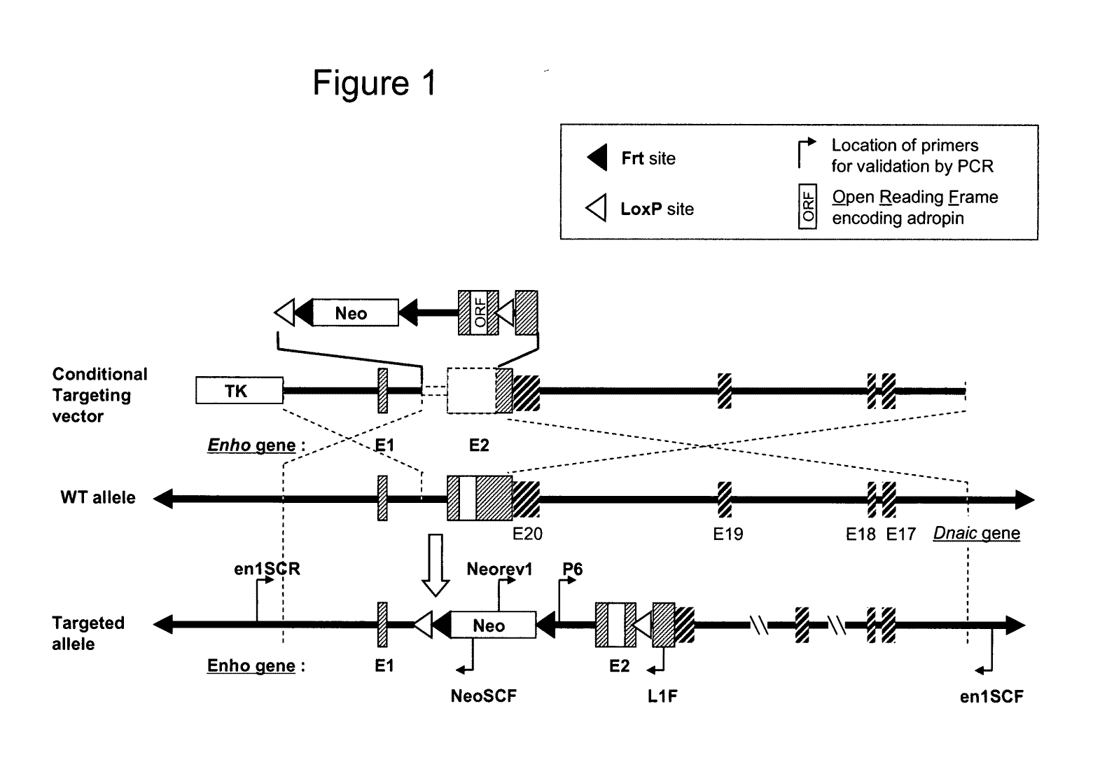

A loxP site with an EcoRV site was inserted into the second exon (E2) downstream of translation termination codon and between the poly(A) sites of the Enho and closely adjacent Dnaic1 genes. A LoxP-Frt-Neo^{r}-Frt sequence was inserted into the first intron via homologous recombination, resulting in exon 2 being flanked by the last fit and loxP sites to generate an Enho targeting construct. The key elements were confirmed by DNA sequencing. This strategy will delete most of exon 2 containing the ADROPIN open reading frame without disrupting the Dnaic1 gene downstream, resulting in complete inactivation of Enho function (FIG. 1).

For gene targeting, 40 μg of Not I-linearized Enho targeting vector (pSW1TV6-1) DNA consisting of 2.5 kb 5′ sequences and 7.2 kb 3′ sequences was electroporated into 1×10⁷ Bruce 4 C57BL/6J embryonic stem (ES) cells (a gift from the National Cancer Institute (NCI)) maintained on mitomycin-C-inactivated STO cells. Transfected ES cells were selected in DMEM medium with 15% fetal bovine serum with 6418 (200 μg/ml).

Correct homologous recombination in targeted clones was identified with Fidelity® PCR at the 5′-end and 3′-end. The primers NeoSCF and en1SCR were used for validating 5′-end recombination event (FIG. 1, left), en1SCF and Neorev1 for validating 3′-end event (FIG. 1, right). The fragments produced from Fidelity PCR with these primers were sequenced to further confirm the correctness of recombination event and the location and sequence of frt and loxP sites. The second loxP was confirmed by sequencing the PCR fragments produced with primers L1F and P6 (FIG. 1).

The targeted ES cells were injected into the blastocysts taken from female Albino C57BL/6J-Tyrc-2J mice (The Jackson Laboratory, Bar Harbor, Me.) to produce chimeric mice. Chimeric mice were mated with Albino C57BL/6J-Tyrc-2J mice, with a black coat color indicating that the offspring descended from the Bruce 4 ES cells. Black C57BL/6J-Tyrc-2J+/− mice that genotyped positively for the presence of the modified gene were then crossed with C57BL/6J mice.

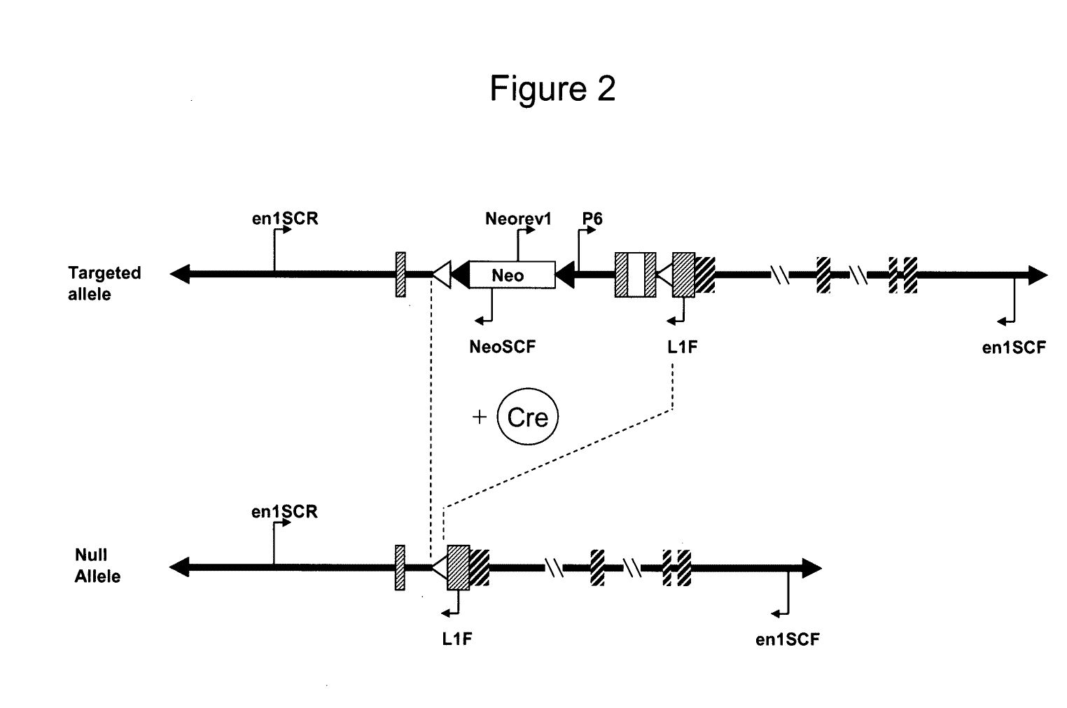

B6.Cg-Enho^{loxP-(frt-Neo-frt)ORF-loxP} were mated with B6.FVB-TgN(EIIa-Cre)C5379 mice purchased from The Jackson Laboratory (Bar Harbor, Me.). The B6.FVB-TgN(EIIa-Cre)C5379 line carries a Cre transgene under the control of the adenovirus Ella promoter, resulting in expression of the Cre protein in the early mouse embryo. Cre-mediated recombination of DNA flanked by LoxP sites (FIG. 2) occurs in a wide range of tissues, including the germ cells that transmit the genetic alteration to progeny. This line was used to create C57BL/6J mice heterozygous for a null Enho allele (B6.Cg-Enho+/−), which were then interbred to generate homozygous Enho knockout mice. The strain on which the various Enho genotypes are maintained, the C57BL/6J mouse, is a classic model of diet-induced obesity and insulin resistance (Collins, S., et al., Physiol Behav., 2004, 81:243-8).

Targeted knockout mice, or mice in which the Enho gene is knocked out in selected tissues rather than in all tissues, are also prepared. B6.Cg-Enho loxP-(frt-Neo-frt)ORF-loxP mice are crossed with B6;SJL-Tg(ACTBFlpe)9205Dym/j to remove Neomycin resistance cassette to produce B6.Cg-Enho^{LoxP2} mice. The B6.Cg-Enho^{LoxP2} mice can be crossed with transgenic mice expressing Cre under the control of tissue/cell-type selective promoters (e.g., liver-specific, brain specific), thus generating mice in which the Enho gene is knocked out in selected tissues such as liver, brain, etc. (see FIG. 2)

### Example 2

### Effect of Enho-Deficient Genotype Upon Fat Mass

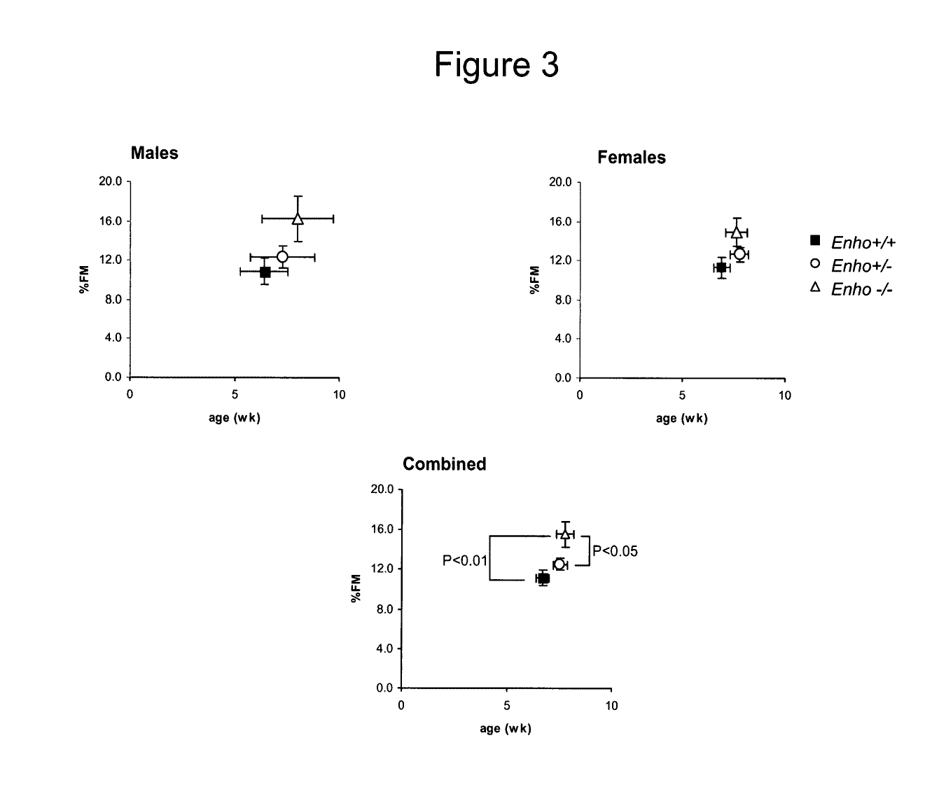

Enho −/−, Enho +/− and Enho +/+pups were weaned from dams at 28 days and fed with mouse breeder chow (Purina 5015, 25% kJ/fat). The genotypes of the pups were determined by carrying out PCR on DNA extracted from blood samples. At approximately 9 weeks of age, the fat mass of the Enho −/−, Enho +/− and Enho +/+mice as a percentage of body weight was determined. Body composition was determined using a Bruker Minispec NMR Analyzer (Bruker Optics, Inc., Billerica, Mass.) and the data analyzed using a 2-way ANOVA accounting for gender and genotype of the mice. The results are summarized in FIG. 3 and the tables below. The Enho−/− mice exhibited a statistically significant greater percent fat mass than wild-type mice.

Twenty-one male mice and 28 female mice were evaluated. The least square means for gender were:

TABLE 1

| | Mean (% fat mass) | SEM |
|---|---|---|
| Male (n = 21) | 13.16 | 0.79 |
| Female (n = 28) | 12.96 | 0.69 |

The least square means for Enho genotype were:

TABLE 2

| | Mean (% fat mass) | SEM | N (m/f) |
|---|---|---|---|
| Enho +/+ | 11.10 | 0.94 | 6 male/9 female |
| Enho +/− | 12.48 | 0.78 | 9 male/12 female |
| Enho −/− | 15.58 | 0.99 | 6 male/7 female |

(P = 0.047 for Enho −/− v Enho +/−)
(P = 0.006 for Enho −/− v Enho +/+)
(P = 0.499 for Enho +/− v Enho +/+)

### Example 3

### Effect of Enho-Deficient Genotype Upon the Expression of Lipogenic Genes

Female Enho −/−, Enho +/− and Enho +/+mice aged 8-9 weeks were fed with mouse breeder chow from weaning at 21 to 24 days of age (Purina 5015, 25% kJ/fat). The expression of a number of genes involved in lipid synthesis (Acetyl coA carboxylase (Acc), Fatty acid synthase (Fasn), Stearoyl-CoA desaturase 1 (Scd1), Sterol regulatory element binding factor 1 (Srebf1)) as well as transport/processing of triglycerides (Lipoprotein lipase (Lpl), Apolipoprotein B (Apob)) in liver tissue was determined in the various Enho −/−, Enho +/− and Enho +/+backgrounds (see Horton, J. D. et al., J. Clin. Invest., 2002, 109:1125-31).

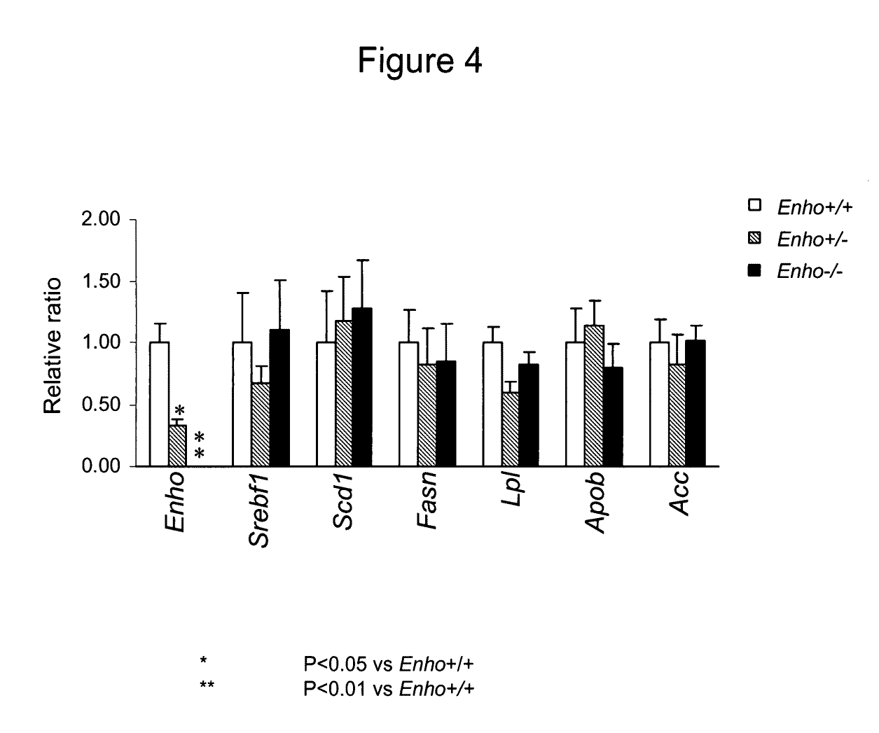

Liver tissues were dissected from the mice and snap frozen in liquid nitrogen followed by storage at −80 C. Total RNA was extracted from the liver samples using either TRI Reagent® or TRIzol® (Invitrogen, Carlsbad, Calif.). The extracted RNA was then reverse transcribed into single stranded DNA using the Superscript III® reverse transcription system (Invitrogen, Carlsbad, Calif.) and gene expression measured by Quantitative RT-PCR. In, some instances, Taqman® Universal PCR Master Mix was used to carry out the RT-PCR in the presence of 6′-Carboxyfluorescein (FAM)-labeled probes synthesized by Applied Biosystems (Foster City, Calif.). In other experiments, SYBR Green® Master Mix was used to carry out the RT-PCR. An ABI PRISM 7900 HT Sequence Detection System® was used to detect fluorescence and determine the level of gene expression. Levels of expression of cyclophilin B were monitored as a reference. Results are shown in FIG. 4 and demonstrate the loss of detectable Enho mRNA in livers of Enho−/− mice; primer pairs, and probes where applicable, were as follows:

TABLE 3

| Gene | Forward Primer (5′->3′) | Reverse Primer (5′->3′) | Probe (5′->3′) |
|---|---|---|---|
| Enho | atggcctcgtaggcttcttg | ggcaggcccagcagaga | tgctactgctctgggtc |
| | (SEQ ID NO: 4) | (SEQ ID NO: 5) | (SEQ ID NO: 6) |
| Srebf1 | aagcgctaccggtcttctatca | gcaagaagcggatgtagtcga | |
| | (SEQ ID NO: 7) | (SEQ ID NO: 8) | |
| Scd1 | caacaccatggcgttcca | ggtgggcgcggtgat | aatgacgtgtacgaatgggcccga |
| | (SEQ ID NO: 9) | (SEQ ID NO: 10) | (SEQ ID NO: 11) |
| Fasn | gtgtgaccgccatctatatc | gtgtcctccttcagcctgtac | ccctgccacccaccgtcagaag |
| | (SEQ ID NO: 12) | (SEQ ID NO: 13) | (SEQ ID NO: 14) |
| Lp1 | tccagccaggatgcaaca | cacgtctccgagtcctctctct | |
| | (SEQ ID NO: 15) | (SEQ ID NO: 16) | |
| Apob | ccagagtgtggagctgaatgtc | cctttcaccatcagactccttg | |
| | (SEQ ID NO: 17) | (SEQ ID NO: 18) | |
| Acc | gacagactgatcgcagagaaag | tggagagccccacacaca | |
| | (SEQ ID NO: 19) | (SEQ ID NO: 20) | |
| CTL | ggtggagagcaccaagacaga | gccggagtcgacaatgatg | ggccgggacaagccactgaaggat |
| | (SEQ ID NO: 21) | (SEQ ID NO: 22) | (SEQ ID NO: 23) |

*Cyclophilin B control

### Example 4

### Effect of Enho-Deficient Genotype Upon the Expression of the Pparg Gene in Retroperitoneal White Adipose Tissue

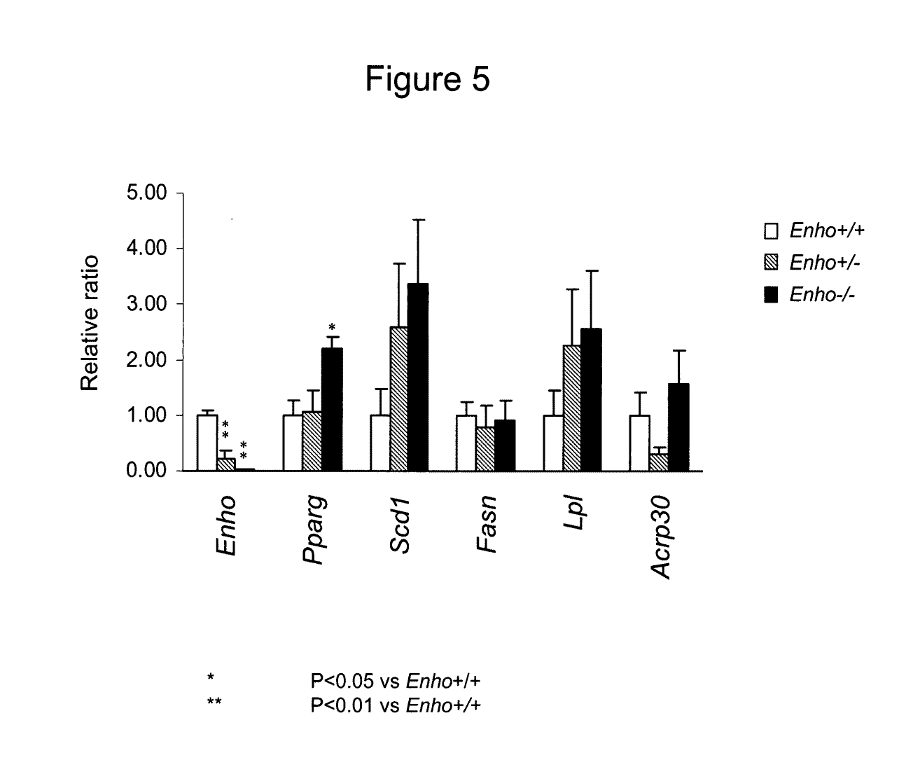

To investigate mechanisms explaining increased adiposity, expression of Pparg mRNA, and expression of genes known to be regulated by Pparg, was measured in adipose tissue. Female Enho −/−, Enho +/− and Enho +/+mice aged 8-9 weeks were fed with mouse breeder chow (Purina 5015, 25% kJ/fat). The expression of the Pparg gene in adipose tissue was determined in the various Enho −/−, Enho +/− and Enho +/+backgrounds. As described in Example 3, RT-PCR was used to measure the levels of mRNA for various genes in white adipose tissue dissected from the various mice. Sequences used for measuring Enho, Scd1, Fasn and Lpl were provided in Example 3. Primer and probe sequences used for quantifying Pparg and adiponectin (Acrp30) mRNA are shown below. The results are summarized in FIG. 5.

TABLE 4

| Gene | Forward Primer (5′->3′) | Reverse Primer (5′->3′) | Probe (5′->3′) |
|---|---|---|---|
| Pparg | gcctatgagcacttcacaagaaatt | gccggagtcgacaatgatg | agccgggacaagccactgaaggat |
| | (SEQ ID NO: 24) | (SEQ ID NO: 25) | (SEQ ID NO: 26) |
| Acrp30 | tggagagccccacacaca | gcccttcagctcctgtcattc | |
| | (SEQ ID NO: 27) | (SEQ ID NO: 28) | |

### Example 5

### Effect of Enho-Deficient Genotype Upon Body Weight, Body Composition and Glucose Metabolism

Male Enho −/−, Enho +/− and Enho +/+mice aged 10-12 weeks were fed a high fat diet (Diet #12492, 60% kJ/fat, Research Diets, New Brunswick, N.J.) for 6 weeks. The mice were housed in cages with wire mesh floors, which allowed for the collection of spillage to more accurately measure food intake. Food intake was monitored as previously described (see Sutton, G. M., et al., Endocrinology, 2006, 147:2183-96).

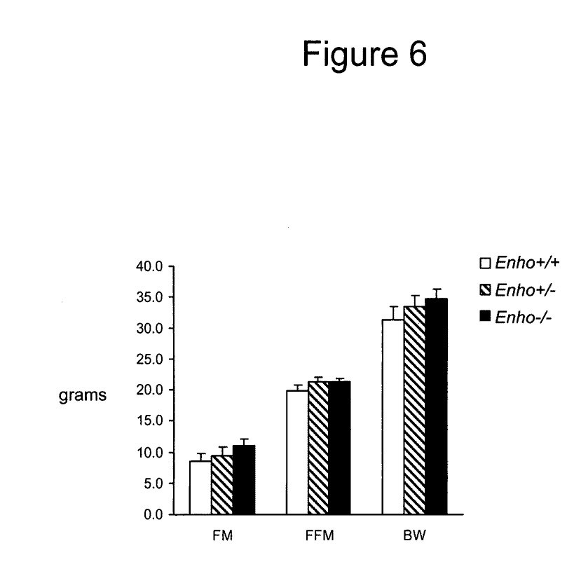

Body weight (in grams) was recorded. Fat mass and fat free mass of Enho −/−, Enho +/− and Enho +/+mice were determined using a Bruker Minispec NMR Analyzer (Bruker Optics, Inc., Billerica, Mass.). The results are summarized in FIG. 6 and in the table below. It was determined that there was no significant difference between food intake, body weight, fat mass and fat-free mass of the mice of Enho −/−, Enho +/− and Enho +/+mice on a high fat diet.

TABLE 5

| | Enho +/+ | Enho +/− | Enho −/− |
|---|---|---|---|
| Gain in fat mass | 6.1 ± 0.9 | 6.2 ± 1.1 | 6.6 ± 1.0 |
| Gain in fat-free mass | 2.6 ± 0.5 | 2.5 ± 0.4 | 2.6 ± 0.5 |
| Gain in body weight | 9.4 ± 1.1 | 9.1 ± 1.5 | 8.9 ± 1.7 |
| Food intake (g/day) | 3.2 ± 0.3 | 3.1 ± 0.1 | 3.3 ± 0.1 |

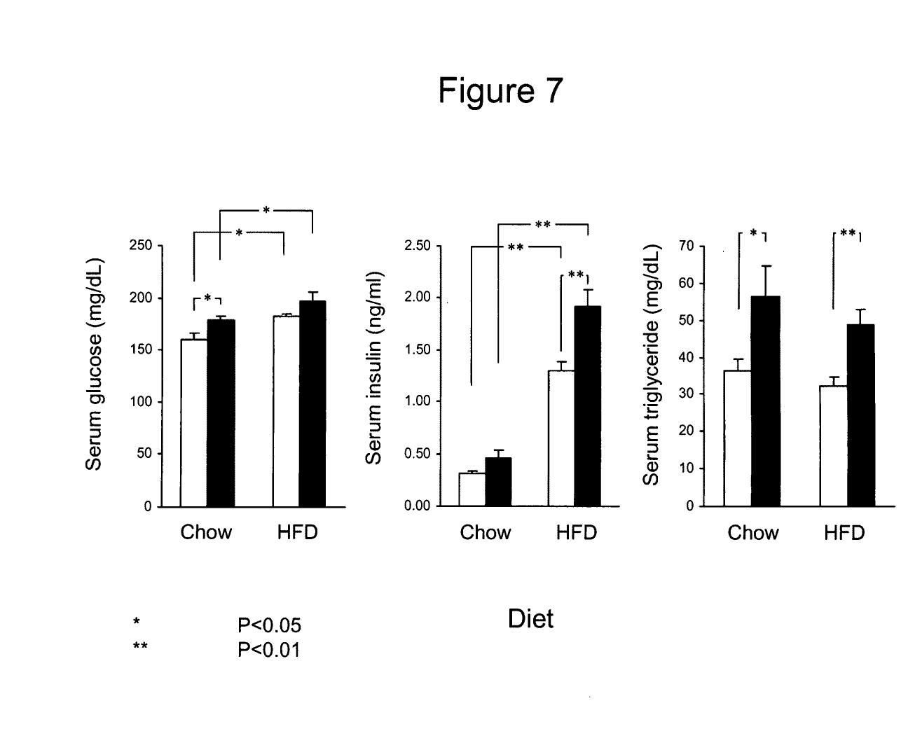

The effect of the presence or absence of ADROPIN upon glucose metabolism was determined by measuring serum glucose and insulin in A) male 3-4 month old Enho+/+ and Enho−/− mice fed standard rodent chow, or B) in male 3-4 month old Enho+/+ and Enho−/− mice after 6 weeks on a high fat diet. In all cases, blood samples were collected after an over night fast. Total serum triglycerides and glucose were measured using a Beckman Synchron CX7® for terminal experiments or by utilizing commercially available kits (for example, see Wako Diagnostics, Richmond, Va.). Insulin was measured using a mouse insulin ELISA kit (Downers Grove, Ill.). These results are shown in FIG. 7.

Data were analyzed using a 2-way Analysis of Variance, with diet and genotype as the independent variables. As noted above, the C57BL/6J mouse strain on which the various Enho genotypes are maintained is a classic model of diet-induced obesity and insulin resistance (Collins, S. et al., Physiol. Behav., 2004, 81:243-8.).

As expected, feeding a high fat diet was associated with a significant increase in fasting glucose (diet effect P<0.01) and insulin (diet effect P<0.001). Serum glucose levels were significantly increased in Enho−/− mice compared to Enho+/+mice (least square means for serum glucose in mg/dL by genotype: Enho+/+171±4; Enho−/−188±4, P<0.01), with no interaction between diet and genotype.

In contrast, the effects of diet on fasting insulin was affected by genotype (interaction between diet and genotype, P<0.05). A statistically significant difference in fasting insulin levels was observed between Enho−/− mice compared to Enho+/+mice maintained on high fat diet. Fasting hypertriglyceridemia, which can be associated with insulin resistance and which contributes to cardiovascular disease (Brown, M. S. et al., Cell Metab., 2008, 7:95-6), was also observed in Enho−/− mice compared to Enho+/+mice, irrespective of diet (effect of genotype, P<0.01; effect of diet, P=0.320).

Observations of hyperinsulinia and hyperglycemia in a fasting subject suggest insulin resistance. To determine whether these phenotypes are associated with glucose intolerance and insulin resistance, glucose clearance tests were conducted.

For glucose tolerance tests, Enho −/−, Enho +/− and Enho +/+mice were fed a high fat diet for 5- to 6-weeks and were fasted overnight prior to the start of testing. At time zero, a blood sample was taken to establish the baseline glucose level. The mice were then given a single intraperitoneal injection of glucose solution (2 mg/kg).

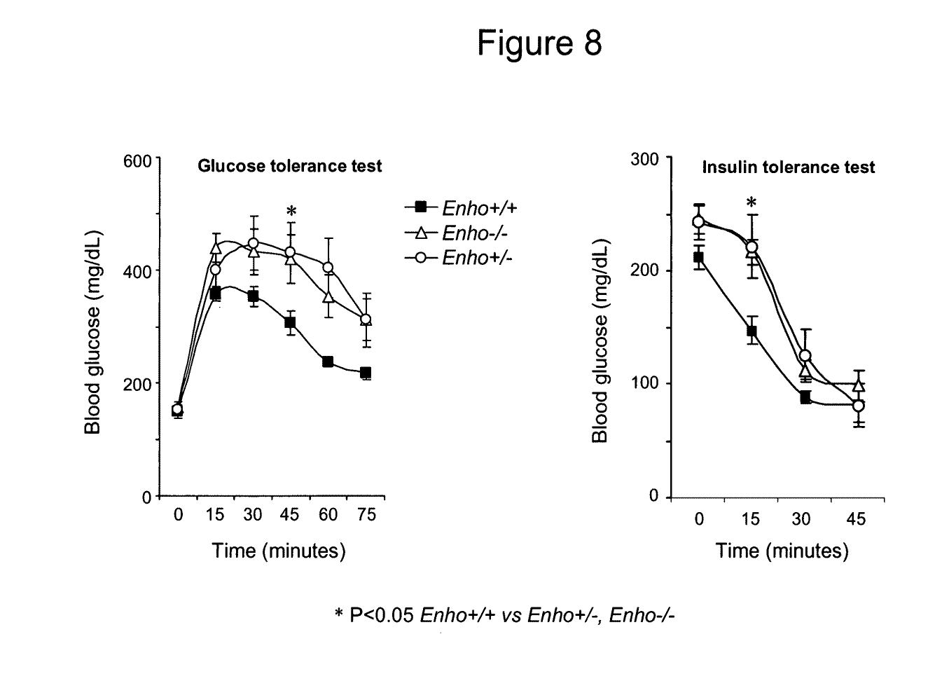

For investigation of glucose clearance in response to insulin, Enho −/−, Enho +/− and Enho +/+mice were fasted for 4 h and then subject to a single injection of 1 U/kg of insulin (Humulin®, Eli Lilly and Co., Indianapolis, Ind.). Blood samples were taken every 15 minutes and the levels of blood glucose were measured using a Glucometer Elite XL® (Bayer Corporation, Elkhart, Ind.). The results are shown in FIG. 8. As can be seen from the data, Adropin deficient mice (Enho −/−, Enho +/− genotypes) exhibit glucose intolerance and reduced clearance of glucose after insulin injection as compared to wild-type (Enho+/+genotype) mice.

Insulin resistance and hypertriglyceridemia are frequently associated with non-alcoholic fatty liver disease (Perlemuter, G. et al. Nat Clin Pract Endocrinol Metab. 2007 3:458-69.) Hematoxylin and eosin staining was performed on 15-μm sections of liver tissue from Enho −/−, Enho +/− and Enho +1+mice fed a high fat diet for 6-weeks collected in 10% paraformaldehyde, dehydrated, and mounted in paraffin. Approximately 100 mg of frozen liver tissue was extracted in a 20-fold volume of 2:1 chloroform:methanol, following which 0.2 volume of methanol was added and the extract vortexed for 30 secs. The mixture was then centrifuged at 1100×g for 10 min and the supernatant collected. A 0.2 volume of 0.04% CaCl₂ was added to the supernatant and then centrifuged at 550×g for 20 min. The upper phase was then removed, and the interface was washed three times with pure solvent upper phase consisting of 1.5 ml of chloroform, 24.0 ml of methanol and 23.5 ml of water. The final wash was removed, and 50 μl of methanol was added to obtain one phase. The samples were then dried under N2 at 60° C. and dissolved in 50 μl of 3:2 tert-butyl alcohol:Triton X-100 (25).

In addition to staining for fat accumulation in the liver tissues, the levels of triglycerides and changes in gene expression in the livers were also determined in Enho−/− and Enho+/+mice fed a high fat diet for 6 weeks. For determining liver lipid content, 0.5 g of liver tissue was homogenized in 10 ml of 2:1 choloroform:methanol solution and filtered through Watman filter paper into a glass centrifuge tube. Approximately, 2.5 ml of 0.9% NaCl was added to the filtered extract which was then vortexed for 30 secs and centrifuged at 1500 RPM for 5 min at 10 C. The upper aqueous phase was then discarded, and 1 ml of 3:47:48 chloroform:methanol:saline (vol:vol:vol) then added and the solution vortexed for 30 secs. Samples were then centrifuged for 5 min at 1500 RPM at 10 C and the lower phase transferred to a new pre-weighed tube. This phase was allowed to air dry inside a fume hood, or flushed with nitrogen to recover to evaporate the organic solvents. The weight of the remaining lipid in the tube was determined by weighing the tube again and then subtracting the original tube weight. For measurement of liver TG, the dried lipid was mixed with 200 ul of ethanol, and 5 ul of the solution used to measure Liver TG using WAKO diagnostic kit as per manufacturer's protocol.

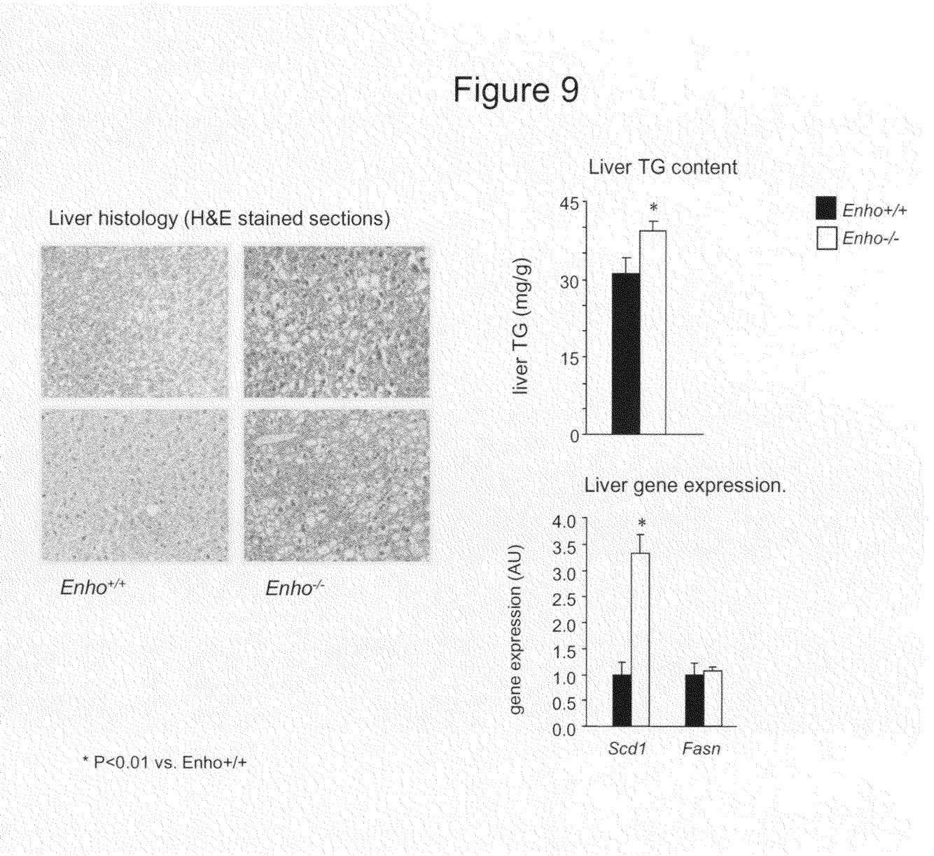

Triglyceride was then quantitated colorimetrically as glycerol using an enzymatic assay. The expression of genes involved in lipid metabolism was determined as described in Example 3. The results are summarized in FIG. 9. As can be seen from the data, Enho−/− mice exhibit a more severe hepatic steatosis, exhibiting altered morphology suggesting accumulation of lipid droplets, a significant increase in triglyceride content of the liver, and increased expression of Scdl mRNA, which expresses an enzyme critical for triglyceride synthesis.

### Example 6

### Effect of Enho-Deficient Genotype on Substrate Metabolism

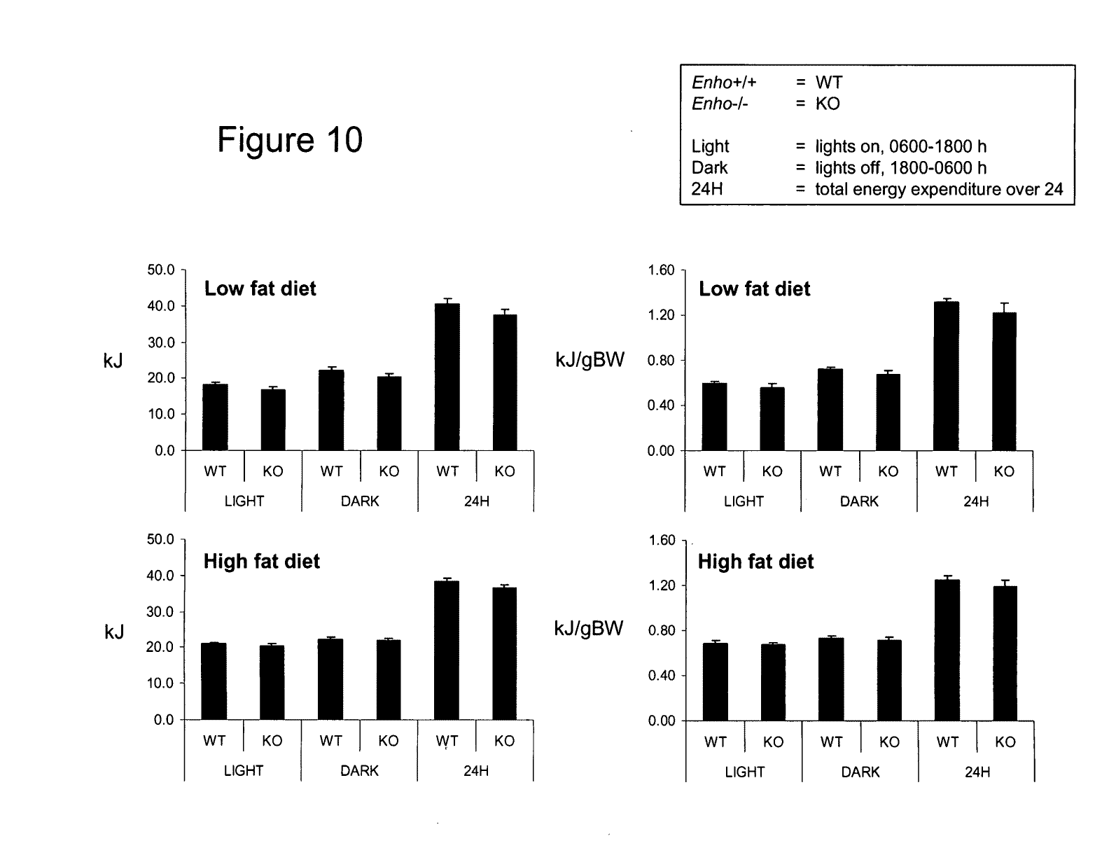

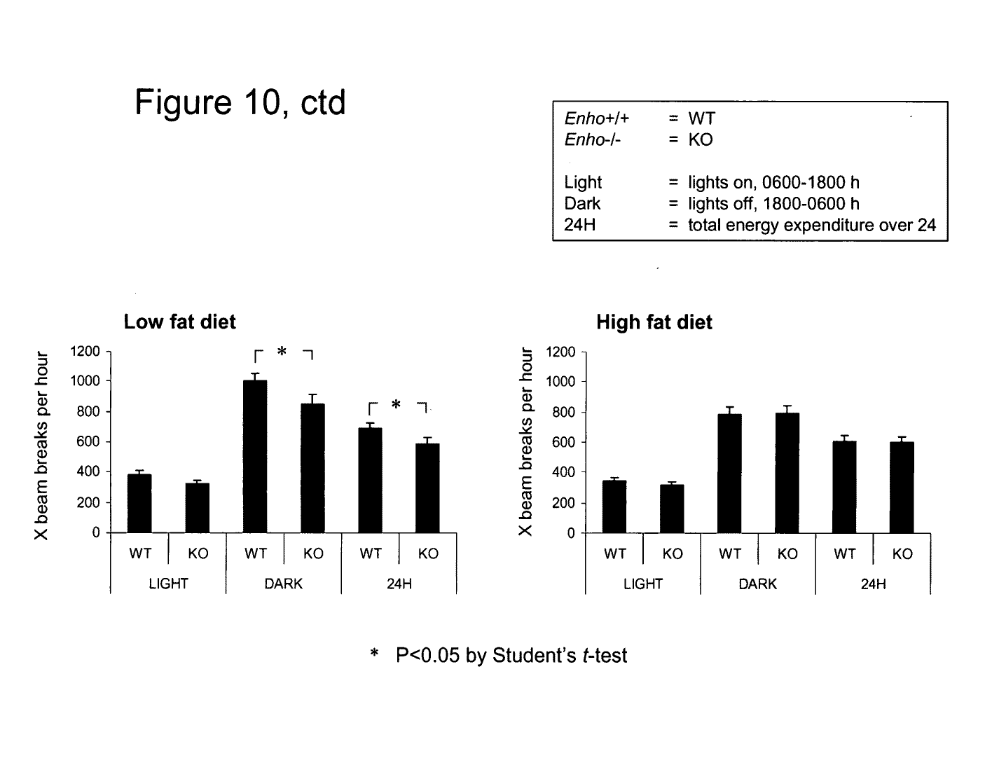

Energy expenditure and whole body substrate metabolism were measured using a comprehensive laboratory animal monitoring system (CLAMS®, Columbus Instruments, Columbus, Ohio) according to established methods (see Sutton G. M. et al., Endocrinology, 2006, 147:2183-96; Butler A. A., Peptides, 2006, 27:281-90). Seven Enho-F/+ and 5 Enho−/− eight-matched male mice aged 3 months were used in the study. Mice were acclimated to housing in the metabolic chambers for 3 days; body weight and food intake parameters were recorded for 6 days. The first three days, the animals were maintained on a low fat diet (10% kJ/fat, 70% kJ/carbohydrate, Research Diet, #12450B) while for the next three days, the animals were maintained on a high fat diet (60% kJ/fat, 20% kJ/carbohydrate, Research Diet, #12492). Data shown represent the mean for 3 days of measurement for each regimen. The body weight and food intake data for the mice used for this experiment are shown below (see also FIGS. 10, 11 and 12). Food intake was measured during high fat intake feeding and showed no difference in food intake.

TABLE 6

| | Enho +/+ (n = 7) | Enho −/− (n = 5) |
|---|---|---|
| Body weight (g) | 30.9 ± 1.2 | 30.9 ± 1.0 |
| Food intake in grams/d | 3.6 ± 0.2 | 3.5 ± 0.2 |
| (high fat diet only) | | |

FIG. 10 shows that there is no statistically significant difference in energy expenditure when assessed as kJ per mouse or adjusted for body weight (kJ/gBW).

FIG. 10 also shows that there was modest (15%) but statistically significant lower level of spontaneous physical activity in Enho−/− mice compared to wild-type controls when fed a low fat diet, but not on a high fat diet.

The respiratory exchange ratio (RER) of the mice was also measured. The RER is the ratio of carbon dioxide exhaled to oxygen inhaled (VCO₂÷VO2); RER can be used as an indicator of the amount of carbohydrate and fat being metabolized to supply the body with energy (see Elia, M. et al., Am. J. Clin. Nutr., 1988, 47:591-607). A low RER suggests a reduced use of carbohydrates and an increased use of fatty acids as a source of energy.

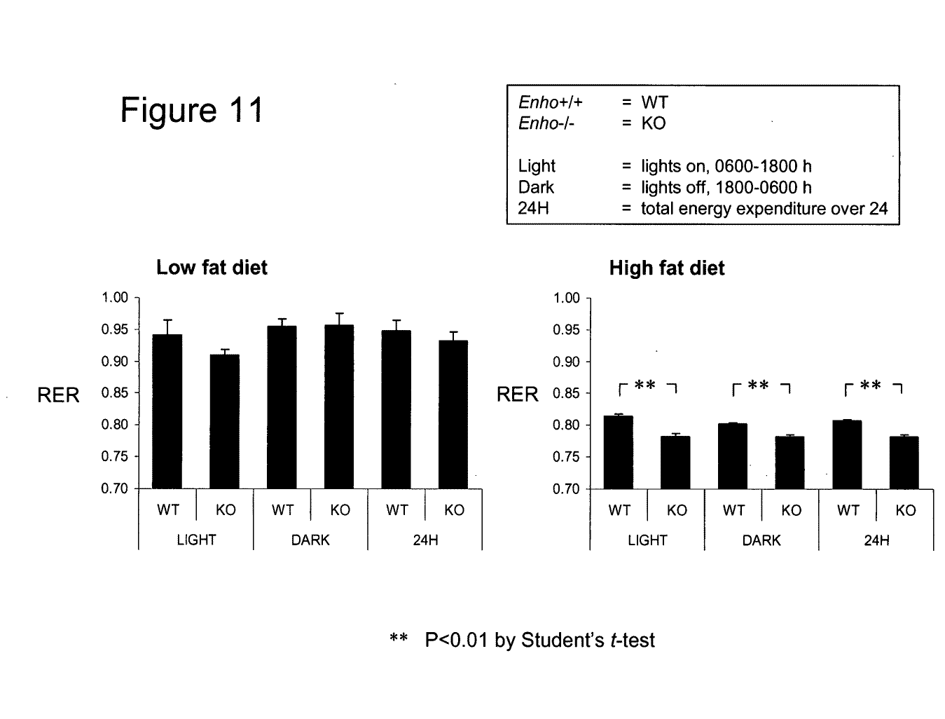

FIG. 11 shows that there was a significant reduction in the RER of Enho−/− mice compared to wild-type controls on a high fat diet. This observation suggests that Enho−/− mice are more dependent on the use of fatty acids for providing energy, perhaps as a consequence of an impaired ability to use glucose. Alternatively, ADROPIN may function to suppress fat oxidation, with loss of ADROPIN in Enho−/− mice leading to a corresponding increase in fat oxidation.

The mice from the metabolic chamber studies were also used to measure the conversion of C¹⁴ palmitic acid to Cain skeletal muscle the Enho−/− and wild-type mice. The amount of CO₂ released reflects the total capacity of red and white muscle to oxidize fatty acids. A description of the method can be found in Sutton, G. M. et al., Endocrinology, 2006, 147:2183-96.

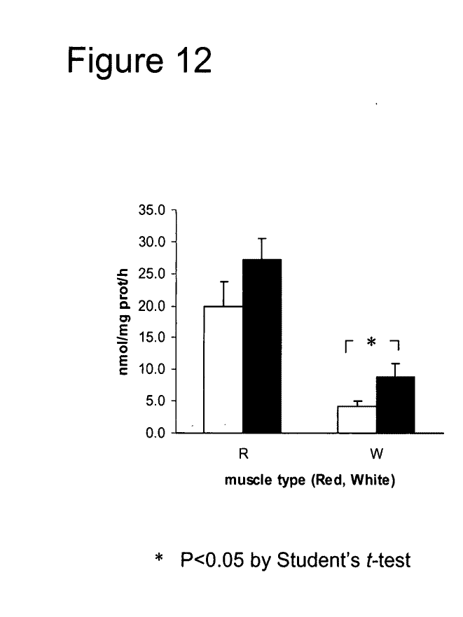

FIG. 12 shows that there is an increase in fat oxidation in the muscle of Enho−/− mice as compared to wild-type controls. In red muscle, there was a trend (P=0.08) for an increase in fatty add oxidation. In white muscle, the increase the conversion of C¹⁴ palmitic acid to CO₂ was statistically significant at P<0.05.

### Example 7

### Use of Enho-Deficient Mice to Study the Diabetic Phenotype

These studies demonstrate that partial or complete loss of ADROPIN function significantly impairs the use of glucose as a metabolic fuel and that such impairment is likely a consequence or causative of insulin resistance. ADROPIN deficient mice exhibit a modest increase in adiposity at an early stage of development. However, neither obesity nor increased calorie intake are the primary cause of deteriorated glucose homeostasis. Glucose intolerance, insulin resistance and altered substrate metabolism were observed in ADROPIN deficient mice after high fat feeding with no significant differences in food intake, body weight or adiposity. Partially ADROPIN deficient Enho +/− mice exhibited comparable glucose intolerance independent of marked obesity.

These observations indicate that ADROPIN plays a fundamental role in regulating glucose and fatty acid metabolism; loss of ADROPIN is associated with a phenotype consistent with diabetes, i.e., glucose intolerance, fasting hyperinsulinemia and fasting hyperglycemia. That glucose intolerance and insulin resistance were also observed with partial ADROPIN deficiency (e.g., Enho +/−) is significant, suggesting that partial loss of function in a population, particularly a human population, may also increase the propensity for metabolic disease.

The Enho+/− and Enho−/− knock-out mice provided by the invention are an excellent model system in which to study the diabetic phenotype. Particular studies of interest include measurements and analysis of:
insulin sensitivity in awake mice using a hyperinsulinemic-euglycemic clamp;
glucose metabolism in muscle and liver using [³H]glucose and 2-[¹⁴C] deoxyglucose;
insulin receptor signal transduction in liver, muscle and white adipose tissue;
pathways involved in glucose and fatty acid uptake in skeletal muscle;
the effect of a synthetic form of ADROPIN (such as Adropin³⁴⁻⁷⁶) to reverse the diabetic phenotype when administered acutely or chronically.

Mice in which the genotype of particular tissues are Enho+/− and Enho−/− are also provided by the invention and are another excellent model system. Of particular interest are studies carried out with mice engineered to contain liver-specific deletions of the Enho gene. As described earlier and show in FIG. 2, Enho^{Lox2/Lox2} mice expressing the Cre recombinase in hepatocytes are used to create liver-specific Enho knockout mice (Enho-LKO). Also as described earlier, PCR and various other molecular biological tools are used to measure Enho mRNA expression in particular tissues of interest, such as the liver. Enho-LKO mice provide a valuable tool to establish whether the regulation of glucose and fatty acid metabolism in the periphery is due to ADROPIN secreted by the liver. Particular studies of interest include measurements and analysis of:
growth curve and body composition (% fat mass, lean mass);
serum insulin, glucose and triglyceride levels in mice fasted overnight followed by assessment of glucose disposal post-injection of glucose or insulin.

### Example 8

### Use of Enho-Deficient Mice to Prepare Antibodies

ADROPIN is a very highly conserved peptide (WO 2007/019426 incorporated herein in its entirety; Kumar K. G., et al., Adropin is a secreted peptide involved in energy homeostasis and lipid metabolism, 2008, submitted to Cell Metabolism, incorporated herein in its entirety) which may make the manufacturing of antibodies for assay development difficult. Homozygous Enho knockout mice (Enho −/−) do not produce ADROPIN and are therefore “naive” and may mount a more robust immune response when challenged with ADROPIN peptides. As such, the invention provides a method of producing antibodies, particularly monoclonal antibodies, utilizing a transgenic mouse, the genome of which comprises a homozygous disruption of the endogenous Enho gene and wherein the transgenic mouse lacks ADROPIN peptide.

Native or synthetic ADROPIN peptides, fragments, analogs, fragments of analogs and derivatives are injected into Enho knockout mice and blood samples drawn and assayed for antibody production. The skilled artisan would know that the ADROPIN peptides and derivatives used for antibody production may be synthesized any number of ways, including but not limited to in vitro synthesis or produced in bacterial, hybridomas or mammalian cells. The peptides are purified before injection by any appropriate method including but not limited to, column chromatography or gel electrophoresis. The antibodies are separated from collected serum and purfied as necessary for use in assays or studies or for detection of ADROPIN in biological samples.

What is claimed:

1. A transgenic mouse, the genome of which comprises a homozygous disruption of the endogenous Enho gene, wherein said transgenic mouse is characterized by reduced ADROPIN peptide activity.

2. The transgenic mouse of claim 1, wherein said disruption is in an intron of the endogenous Enho gene.

3. The transgenic mouse of claim 2, wherein said disruption is a deletion of a portion of said intron.

4. The transgenic mouse of claim 2, wherein said disruption is a point mutation in said intron.

5. The transgenic mouse of claim 1, wherein said disruption is in an exon of the endogenous Enho gene.

6. The transgenic mouse of claim 5, wherein said disruption is a deletion of a portion of said exon.

7. The transgenic mouse of claim 5, wherein said disruption is a deletion of exon 2.

8. The transgenic mouse of claim 7, wherein said disruption is a deletion of a portion of exon 2.

9. The transgenic mouse of claim 5, wherein said disruption is a point mutation in said exon.

10. The transgenic mouse of claim 9, wherein said disruption is a point mutation in exon 2.

11. The transgenic mouse of claim 1, wherein said disruption of the endogenous Enho gene is prepared using Cre-lox technology.

12. The transgenic mouse of claim 1, wherein said mouse is resistant to insulin.

13. The transgenic mouse of claim 1, wherein said mouse exhibits glucose intolerance.

14. The transgenic mouse of claim 1, wherein said mouse exhibits glucose intolerance on a high fat diet.

15. The transgenic mouse of claim 1, wherein said mouse exhibits increased adiposity.

16. The transgenic mouse of claim 1, wherein said mouse exhibits non-alcoholic fatty acid liver disease.

17. The transgenic mouse of claim 1, wherein said mouse exhibits increased expression of the Pparg gene in adipose tissue.

18. The transgenic mouse of claim 1, wherein said mouse exhibits increased expression of the Pparg gene in adipose tissue.

19. The use of the transgenic mouse of claim 1 to study the regulation of conditions related to ADROPIN peptide activity.

20. The use of the transgenic mouse of claim 1 to study insulin resistance.

21. The use of the transgenic mouse of claim 1 to study the regulation of insulin.

22. The use of the transgenic mouse of claim 1 to study the regulation of glucose

23. The use of the transgenic mouse of claim 1 to study ADROPIN function in mammalian systems.

24. The use of the transgenic mouse of claim 1 to study the regulation of lipid biochemistry in mammalian systems.

25. The use of the transgenic mouse of claim 1 to produce hybridomas expressing monoclonal antibodies recognizing native ADROPIN, fragments of native ADROPIN, analogs of ADROPIN or fragments of analogs of ADROPIN.

26. The transgenic mouse of claim 1, wherein said transgenic mouse lacks detectable ADROPIN peptide activity.

27. A transgenic mouse, the genome of which comprises a heterozygous disruption of the endogenous Enho gene, wherein said transgenic mouse is characterized by reduced ADROPIN peptide activity.

28. The transgenic mouse of claim 27, wherein said disruption is in an intron of the endogenous Enho gene.

29. The transgenic mouse of claim 28, wherein said disruption is a deletion of a portion of said intron.

30. The transgenic mouse of claim 28, wherein said disruption is a point mutation in said intron.

31. The transgenic mouse of claim 27, wherein said disruption is in an exon of the endogenous Enho gene.

32. The transgenic mouse of claim 31, wherein said disruption is a deletion of a portion of said exon.

33. The transgenic mouse of claim 31, wherein said disruption is a deletion of exon 2.

34. The transgenic mouse of claim 33, wherein said disruption is a deletion of a portion of exon 2.

35. The transgenic mouse of claim 31, wherein said disruption is a point mutation in said exon.

36. The transgenic mouse of claim 35, wherein said disruption is a point mutation in exon 2.

37. The transgenic mouse of claim 27, wherein said disruption of the endogenous Enho gene is prepared using Cre-lox technology.

38. The transgenic mouse of claim 27, wherein said mouse is resistant to insulin.

39. The transgenic mouse of claim 27, wherein said mouse exhibits glucose intolerance.

40. The transgenic mouse of claim 27, wherein said mouse exhibits glucose intolerance on a high fat diet.

41. The transgenic mouse of claim 27, wherein said mouse exhibits increased adiposity.

42. The transgenic mouse of claim 27, wherein said mouse exhibits non-alcoholic fatty acid liver disease.

43. The transgenic mouse of claim 27, wherein said mouse exhibits increased expression of the Pparg gene in adipose tissue.

44. The transgenic mouse of claim 27, wherein said mouse exhibits increased expression of the Pparg gene in adipose tissue.

45. The use of the transgenic mouse of claim 27 to study the regulation of conditions related to ADROPIN peptide activity.

46. The use of the transgenic mouse of claim 27 to study insulin resistance.

47. The use of the transgenic mouse of claim 27 to study the regulation of insulin.

48. The use of the transgenic mouse of claim 27 to study the regulation of glucose

49. The use of the transgenic mouse of claim 27 to study ADROPIN function in mammalian systems.

50. The use of the transgenic mouse of claim 27 to study the regulation of lipid biochemistry in mammalian systems.


SEQUENCE LISTING (28 sequences)

SEQ ID NO: 1 - DNA, 868 bp, mus sp.
cccgttgtcccggaccctctcgcgggcgcgcaccgggctcaactcaggcccaggactgcaggtgggcatcttccctgccaagaagtcgctgtgtgtggacaggacagccaccttggatggttggccaccccagagttgtgcctcggcatgggccttgccgctgaggcagctccactgtctgcgctggcctgagggtgctgtctgtcatgggggcagccatctcccaaggggctctcatcgccatcgtctgcaatggcctcgtaggcttcttgctgctactgctctgggtcattctctgctgggcctgccattctcgatctgctgacgtcgattctctctcggaatccagtcccaactccagccctggcccctgtcctgagaaggcgccaccaccccagaagcccagccatgaaggcagctacctgctgcagccctgaagggctctggcctagcctggagtcctggacctgagtatacctgagtcagagcgtggaatcggatccaagaagtcagtcggcctggggtccagtcgatttgacactggacccagcagcctagattgtagccagcctggctccaagagaggcctgagtggccctagagagaaaggcctggagggggggttaggagttggtgctagggccagggccatctggactctgctccatcccaagggccaagggctgagtccatgccttccctaggctcagcacatctgggctccctaggttggggagcaaacgggaaccccatggcaataatgggagggtgtccaggctgggccccttctctggtcctcccactgtttgttgggtaataaatggaactatggcttgcaaaaaaaaaaaaaaaaaaaaaaaaaaaaaaa

SEQ ID NO: 2 - protein, 76 aa, mus sp.
MGAAISQGALIAIVCNGLVGFLLLLLWVILCWACHSRSADVDSLSESSPNSSPGPCPEKAPPPQKPSHEGSYLLQP

SEQ ID NO: 3 - protein, 43 aa, Mus sp.
CHSRSADVDSLSESSPNSSPGPCPEKAPPPQKPSHEGSYLLQP

SEQ ID NO: 4 - DNA, 20 bp, Artificial
  Synthetic oligonucleotide
atggcctcgtaggcttcttg

SEQ ID NO: 5 - DNA, 17 bp, Artificial Sequence
  Synthetic oligonucleotide
ggcaggcccagcagaga

SEQ ID NO: 6 - DNA, 17 bp, Artificial Sequence
  Synthetic oligonucleotide
tgctactgctctgggtc

SEQ ID NO: 7 - DNA, 22 bp, Artificial Sequence
  Synthetic oligonucleotide
aagcgctaccggtcttctatca

SEQ ID NO: 8 - DNA, 21 bp, Artificial Sequence
  Synthetic oligonucleotide
gcaagaagcggatgtagtcga

SEQ ID NO: 9 - DNA, 18 bp, Artificial Sequence
  Synthetic oligonucleotide
caacaccatggcgttcca

SEQ ID NO: 10 - DNA, 15 bp, Artificial Sequence
  Synthetic oligonucleotide
ggtgggcgcggtgat

SEQ ID NO: 11 - DNA, 24 bp, Artificial Sequence
  Synthetic oligonucleotide
aatgacgtgtacgaatgggcccga

SEQ ID NO: 12 - DNA, 20 bp, Artificial Sequence
  Synthetic oligonucleotide
gtgtgaccgccatctatatc

SEQ ID NO: 13 - DNA, 21 bp, Artificial Sequence
  Synthetic oligonucleotide
gtgtcctccttcagcctgtac

SEQ ID NO: 14 - DNA, 22 bp, Artificial Sequence
  Synthetic oligonucleotide
ccctgccacccaccgtcagaag

SEQ ID NO: 15 - DNA, 18 bp, Artificial Sequence
  Synthetic oligonucleotide
tccagccaggatgcaaca

SEQ ID NO: 16 - DNA, 22 bp, Artificial Sequence
  Synthetic oligonucleotide
cacgtctccgagtcctctctct

SEQ ID NO: 17 - DNA, 22 bp, Artificial Sequence
  Synthetic oligonucleotide
ccagagtgtggagctgaatgtc

SEQ ID NO: 18 - DNA, 22 bp, Artificial Sequence
  Synthetic oligonucleotide
cctttcaccatcagactccttg

SEQ ID NO: 19 - DNA, 22 bp, Artificial Sequence
  Synthetic oligonucleotide
gacagactgatcgcagagaaag

SEQ ID NO: 20 - DNA, 18 bp, Artificial Sequence
  Synthetic oligonucleotide
tggagagccccacacaca

SEQ ID NO: 21 - DNA, 21 bp, Artificial Sequence
  Synthetic oligonucleotide
ggtggagagcaccaagacaga

SEQ ID NO: 22 - DNA, 19 bp, Artificial Sequence
  Synthetic oligonucleotide
gccggagtcgacaatgatg

SEQ ID NO: 23 - DNA, 24 bp, Artificial Sequence
  Synthetic oligonucleotide
ggccgggacaagccactgaaggat

SEQ ID NO: 24 - DNA, 25 bp, Artificial Sequence
  Synthetic oligonucleotide
gcctatgagcacttcacaagaaatt

SEQ ID NO: 25 - DNA, 19 bp, Artificial Sequence
  Synthetic oligonucleotide
gccggagtcgacaatgatg

SEQ ID NO: 26 - DNA, 24 bp, Artificial Sequence
  Synthetic oligonucleotide
agccgggacaagccactgaaggat

SEQ ID NO: 27 - DNA, 18 bp, Artificial Sequence
  Synthetic oligonucleotide
tggagagccccacacaca

SEQ ID NO: 28 - DNA, 21 bp, Artificial Sequence
  Synthetic oligonucleotide
gcccttcagctcctgtcattc